(19) United States
(12) Patent Application Publication
(10) Pub. No.: US 2026/0224372 A1
(43) Pub. Date: Aug. 6, 2026

(54) EXPANDABLE ARTICULATING TRANSFORAMINAL LUMBAR INTERBODY SPACER

(71) Applicant: Astura Medical Inc., Irving, TX (US)

(72) Inventor: Thomas Prucell, Irving, TX (US)

(73) Assignee: Astura Medical Inc., Irving, TX (US)

(21) Appl. No.: 19/531,332

(22) Filed: Feb. 5, 2026

Related U.S. Application Data
(60) Provisional application No. 63/754,117, filed on Feb. 5, 2025.

(51) Int. Cl. A61F 2/44; A61F 2/30

(52) U.S. Cl. CPC A61F 2/447; A61F 2002/30528; A61F 2002/30538; A61F 2002/30579

(57) ABSTRACT

An expandable lateral lumbar interbody spacer for placement between adjacent vertebrae configured to have an initial collapsed state suitable for being inserted into an intervertebral space defined by a pair of adjacent vertebrae, and a final expanded state by expanding the expandable interbody spacer in sequential steps: first lateral expansion, then lordotic expansion, then linear expansion.

### CROSS-REFERENCE TO RELATED APPLICATIONS

This application claims the benefit of U.S. Provisional Application No. 63/754,117, titled EXPANDABLE ARTICULATING TRANSFORAMINAL LUMBAR INTERBODY SPACER, filed Feb. 5, 2025, which is incorporated herein by reference.

### FIELD

The present invention relates generally to the field of surgery, and more specifically, to an expandable intervertebral body fusion device (i.e., “interbody spacer”) for placement in intervertebral space between adjacent vertebrae.

### BACKGROUND

A spinal disc can become damaged as a result of degeneration, dysfunction, disease and/or trauma. Conservative treatment can include non-operative treatment through exercise and/or pain relievers to deal with the pain. In surgical treatments, interbody spacers may be used between adjacent vertebra, resulting in spinal fusion of the adjacent vertebra. Treatment options include disc removal and replacement using an interbody spacer such as anterior cervical interbody fusion (ACIF), anterior lumbar interbody fusion (ALIF), direct lateral interbody fusion (DLIF) (also known as XLIF), posterior lumbar interbody fusion (PLIF), and transforaminal lumbar interbody fusion (TLIF).

A fusion is a surgical method wherein two or more vertebrae are joined together (fused) by way of interbody spacers, sometimes with bone grafting, to form a single bone. The current standard of care for interbody fusion requires surgical removal of all or a portion of the intervertebral disc. After removal of the intervertebral disc, the interbody spacer is implanted in the interspace.

Interbody spacers must be inserted into the intervertebral space in the same dimensions as desired to occupy the intervertebral space after the disc is removed. This requires that an opening sufficient to allow the interbody spacer must be created through surrounding tissue to permit the interbody spacer to be inserted into the intervertebral space. In some cases, the intervertebral space may collapse prior to insertion of the interbody spacer. In these cases, additional hardware may be required to increase the intervertebral space prior to insertion of the implant.

In addition, minimally invasive surgical techniques may have been used on the spine. Under minimally invasive techniques, small incisions are done to access the intervertebral space. Through these incisions, discs are removed, and an interbody spacer is placed in the intervertebral disc space to restore normal disc height. Minimally invasive spine surgery offers multiple advantages as compared to open surgery. Advantages include: minimal tissue damage, minimal blood loss, smaller incisions and scars, minimal post-operative discomfort, and relative quick recovery time and return to normal function.

Typical expandable implants utilize angled ramps in conjunction with slots or t-rails and a separate drive screw to compress the mechanism and force expansion. This type of mechanism limits the available expansion of the implant due to the amount of material required to house the slots or t-rails.

The expandable implants are typically expensive and difficult to manufacture due to the complexity of the expansion mechanism and the available manufacturing techniques require the size and positional tolerance of these features to be relatively loose to allow for manufacturability.

It would be desirable to develop an easy to manufacture expandable interbody spacer that can be inserted into the intervertebral space at a first smaller dimension and once in place, deploy to a second, larger dimension.

### SUMMARY

Disclosed is an expandable interbody spacer that is configured to have an initial collapsed state suitable for being inserted into an intervertebral space defined by a pair of adjacent vertebrae, and a final expanded state by expanding the expandable interbody spacer in steps: first lateral expansion, then lordotic expansion, then linear/vertical expansion. The expandable articulating transforaminal lumbar interbody spacer can achieve 25 degrees of lordosis/Endplate angulation. The expandable interbody spacer may be expanded from the initial collapsed state to the expanded state in-situ. The expanded state increases the distance between the adjacent vertebrae and provides support to the adjacent vertebrae while bone fusion occurs and also provides rigid support between the adjacent vertebrae that withstands compressive forces. By inserting the expandable interbody spacer into the intervertebral space in the initial collapsed state, it is possible to perform the surgery percutaneously with minimal disruption to tissues surrounding the surgical site and intervening soft tissue structures.

### BRIEF DESCRIPTION OF THE DRAWINGS

FIG. 1 is a perspective view of an expandable articulating transforaminal lumbar interbody spacer in a collapsed state.

FIG. 2 is a perspective view showing the expandable interbody spacer of FIG. 1 in an expanded state.

FIG. 3 is a perspective view of FIG. 2 with an endplate removed to show more details of the components.

FIGS. 4A, 4b show a top view and a side view of the expandable interbody spacer in the collapsed state.

FIGS. 5A, 5B show a top view and a side view of the expandable interbody spacer in the expanded state.

FIGS. 6A-6C are sectional top views showing lateral expansion of the expandable interbody spacer.

FIGS. 7A-7C are sectional side views showing lordotic expansion of the expandable interbody spacer.

FIGS. 8A, 8B are sectional side views showing linear/vertical expansion of the expandable interbody spacer.

FIGS. 9A, 9B are sectional side views of the expandable interbody spacer 100 in the collapsed and expanded states

### DETAILED DESCRIPTION

The interbody spacer disclosed is for a transforaminal lumbar interbody fusion (TLIF) but the same features may be used for anterior cervical interbody fusion (ACIF), anterior lumbar interbody fusion (ALIF), direct lateral interbody fusion (DLIF) (also known as XLIF), and posterior lumbar interbody fusion (PLIF). The expandable interbody spacer includes a collapsed state and expanded state. The collapsed state allows insertion between the adjacent vertebrae with minimal dimensions.

An expandable articulating transforaminal lumbar interbody spacer is shown that expands in width first (lateral expansion), then lordosis (lordotic expansion), then height (linear/vertical expansion). The expandable articulating transforaminal lumbar interbody spacer can achieve 25 degrees of lordosis/endplate angulation.

The unique aspect of this device is the configuration of the ramps that allows for lateral expansion, then lordotic expansion, then linear/vertical expansion. The key mechanisms are the expansion limiters due to their ability to stop lateral translation and transition to linear translation. The lateral wedge/component is a key difference in the way that it houses the vertical expansion wedges within slots or cavities. Since the vertical expansion wedges are positioned in the slots or cavities of the lateral expansion wedges, they are also laterally translated with the lateral expansion wedges.

For Lateral Expansion, the frame and lateral expansion wedges contain angled ramps that induce lateral expansion when a drive screw is initially rotated. The rotation of the drive screw contacts the threads within the housing and translates it towards the distal end, which forces the lateral expansion wedge and vertical wedge outward. Lateral expansion stops when lateral expansion limiting balls reach the end of limiting groove slots on the ramps.

For Lordosis/Angular Expansion, once the lateral expansion reaches its limit, the Drive Screw continues turn, further translating the housing towards the distal end. At the point when the lateral expansion reaches its limit, the housing begins translating the vertical expansion wedges towards the distal end. At this point the ramps of the vertical expansion wedges contact the ramps of the endplates which force them into the lateral housing face and distal end creating a counterforce and then they ultimately translate upward. There is a difference in angle between the two ramps which creates the angular/lordotic expansion.

For Linear/Vertical Expansion, if the screw is continued to be turned, the angle of the endplates will reach a point where their ramp angle matches the angle of the vertical expansion wedges. When this point is reached, the lordosis/angular expansion of the endplates stops and the endplates transition to a linear/vertical expansion.

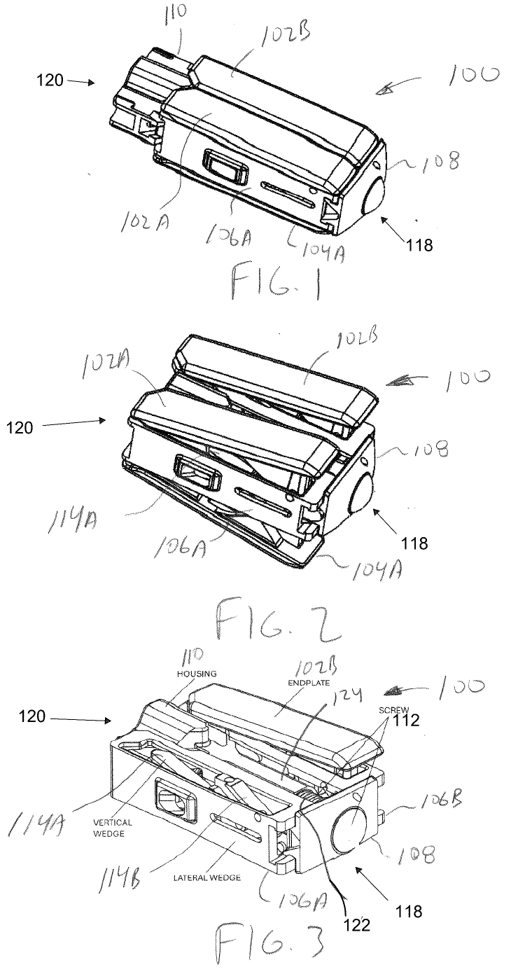

FIGS. 1 and 2 are perspective views showing an expandable articulating transforaminal lumbar interbody spacer 100 (“expandable interbody spacer 100”) the includes: a frame, a housing, two upper endplates, two lower endplates, right and left lateral expansion wedges, and a drive screw coupled to the frame and housing. The unique design of the expandable interbody spacer 100 allows it to expand in sequential steps, starting with lateral expansion or translation (width expansion) of the left and right lateral expansion wedges away from each other, then lordotic expansion (lordotic angle) of the distal end of the upper and lower endplates, and finally, linear/vertical expansion (height expansion) of the upper and lower endplates while maintaining the lordotic angle. Each of the steps is described below.

One of the key features of the expandable interbody spacer during expansion is the use of expansion limiters due to their ability to stop lateral translation of the left and right lateral expansion wedges, and transition to linear translation of the upper and lower endplates. The lateral wedge/component is a key difference in the way that it houses the vertical expansion wedge allowing for lateral and orthogonal translation.

Referring again to FIG. 1, the expandable interbody spacer 100 is shown in a collapsed state having right and left upper endplates 102A, 102B, right and left lower endplates 104A, 104B, left and right lateral expansion wedges 106A, 106B, a frame 108 and a housing 110. The frame 108 is positioned on a distal end or front end of the expandable interbody spacer 100, and the housing 110 is positioned on a proximal end or rear end of the expandable interbody spacer 100. A screw

Referring again to FIG. 2, the expandable interbody spacer 100 is shown in an expanded state after lateral expansion of the lateral expansion wedges, lordotic expansion of the distal end of the upper and lower endplates, and linear/vertical expansion upper and lower endplates. During expansion from the collapsed state to the expanded state includes: 1) lateral expansion: the right and left lateral expansion wedges 106A, 106B expand away from each and increase the width of the expandable interbody spacer 100; 2) lordotic expansion: the distal ends of the upper endplates 102A, 102B and lower endplates 104A, 104B expand away from each other to create a lordotic angle; and 3) linear/vertical expansion: the upper endplates 102A, 102B and lower endplates 104A, 104B linearly expand away from each other increasing the height expandable interbody spacer 100.

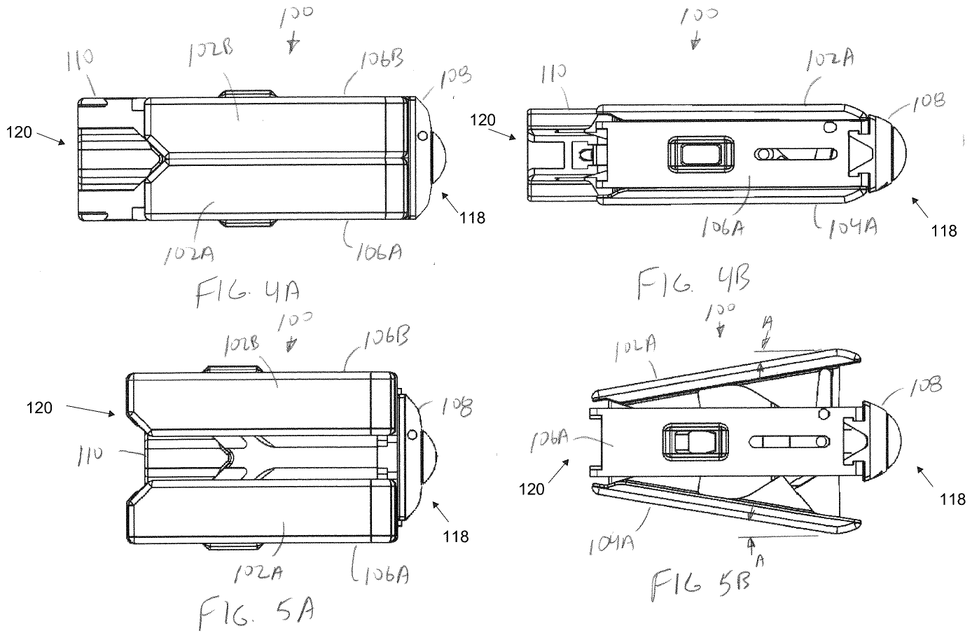

FIG. 3 is a perspective view of FIG. 2 with an upper endplate removed to show some more detail of the expansion components, including left and right lateral wedges 106A, 106B having angled ramps configured to engage the housing 110 and frame 108, and proximal and distal vertical expansion wedges 114A, 114B configured to engage the upper and lower endplates 102A, 102B, 104A, 104B, and a threaded portion 124 of the housing 110 configured to engage a threaded portion 122 of the drive screw 112. The distal vertical expansion wedge 114B has a steeper or greater angle than the proximal vertical expansion wedge 114A.

The lateral wedges 106A, 106B have angled ramps that induce lateral expansion when the drive screw is initially rotated. The rotation of the drive screw 112 translates the housing 110 towards the distal end 118, which forces the lateral wedges 106A, 106B outward. After lateral expansion, the drive screw 112 continues to rotate and moves the housing 110 toward the distal end 118 and the distal vertical expansion wedge 114B engage and translate the distal end of the upper and lower endplates 102A, 102B, 104A, 104B, resulting in lordotic expansion of the upper and lower endplates 102A, 102B, 104A, 104B. When lordotic expansion is complete, the drive screw 112 continues to rotate and the proximal and distal vertical expansion wedges 114A, 114B expand the upper and lower endplates 102A, 102B, 104A, 104B.

FIGS. 4A, 4b show a top view and a side view of the expandable interbody spacer 100 in the collapsed state.

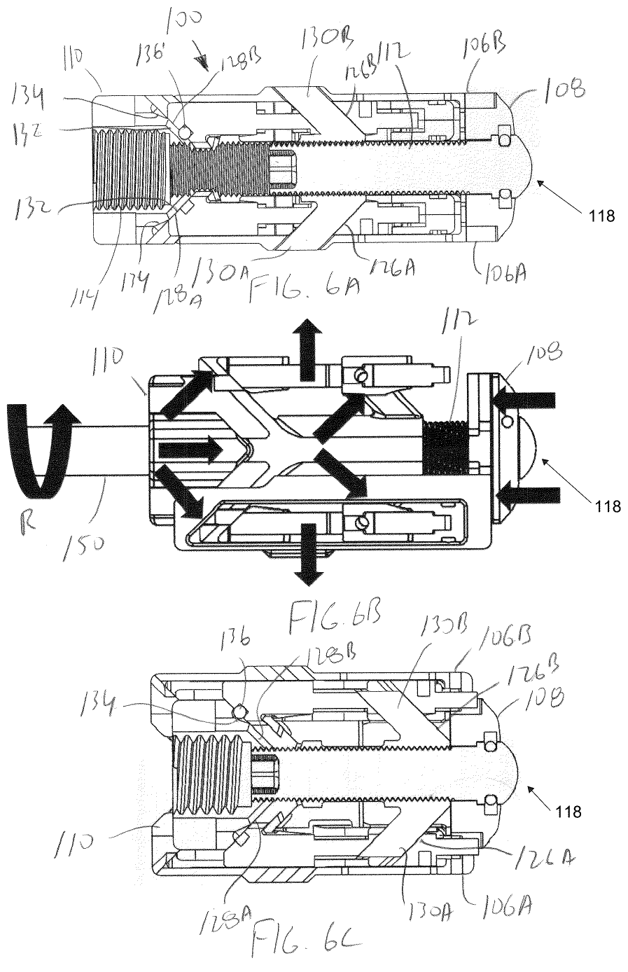

FIGS. 5A, 5B show a top view and a side view of the expandable interbody spacer 100 in the expanded state.

Expansion of the expandable interbody spacer 100 includes 1) lateral expansion, 2) lordotic expansion, and 3) linear/vertical expansion. During lateral expansion, the right and left lateral expansion wedges 106A, 106B move laterally with the upper and lower endplates 102A, 102B, 104A, 104B. During lordotic expansion, the distal vertical expansion wedge 114B engage the distal end of the upper and lower endplates 102A, 102B, 104A, 104B. creating a lordotic angle. During linear/vertical expansion, the proximal and distal vertical expansion wedges 114A, 114B engage the upper and lower endplates 102A, 102B, 104A, 104B increasing the height of the expandable interbody spacer 100.

The proximal end of the drive screw 112 includes a threaded portion 114 configured to engage one or more tools, such as an actuation tool or actuation device 150. The various tools may also include insertion tools with various attachment features to enable percutaneous insertion of the expandable interbody spacer 100 and/or removal tools. During use, the expandable interbody spacer 100 may be inserted and positioned within the intervertebral space between two vertebrae with the insertion tool, the actuation device 150 expands the expandable interbody spacer 100 by applying a rotational force to the drive screw 112.

During expansion, the expandable interbody spacer 100 expands in steps, starting with lateral expansion (width), then lordotic expansion (lordotic angle), then linear/vertical expansion (height). The key mechanisms during expansion are expansion limiters due to their ability to stop lateral translation and transition to linear translation. The lateral wedge/component is a key difference in the way that it houses the vertical expansion wedge allowing for lateral and orthogonal translation. Each of the steps are described below.

### Lateral Expansion

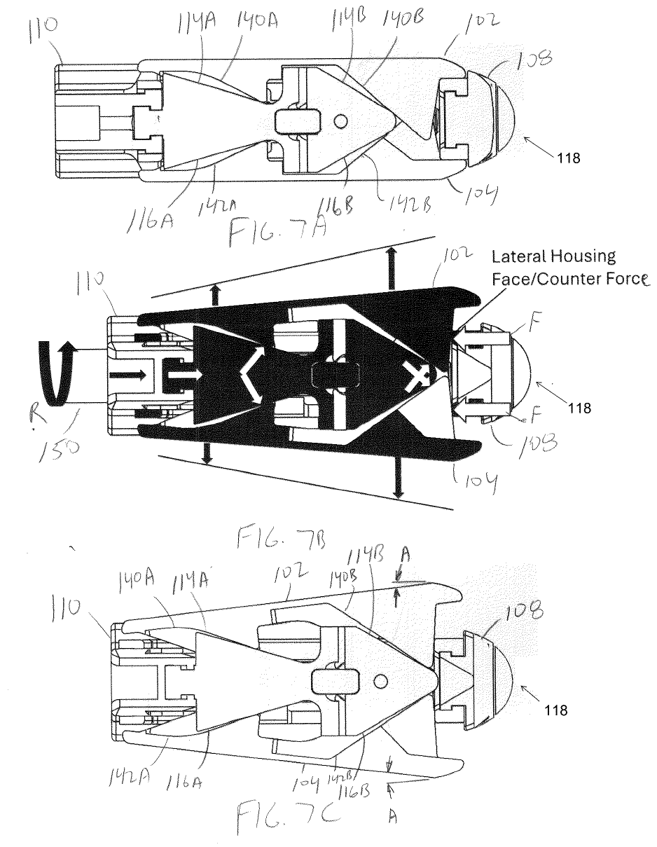

FIGS. 6A-6C are sectional top views showing lateral expansion of the expandable interbody spacer 100. The frame, lateral expansion wedges, and vertical expansion wedges all contain angled ramps that induce lateral expansion when the drive screw is initially rotated.

FIG. 6A is a sectional top view showing the expandable interbody spacer 100 in a collapsed state. The housing includes lateral expansion ramps 130, 132 configured to engage lateral wedge ramps 126, 128 in the lateral wedges 106A, 106B. There are also limiting grooves 134 and limiting balls 136 configured to stop the lateral movement of the lateral wedges 106A, 106B when lateral expansion is complete. One of the unique aspects of the expandable interbody spacer 100 is the way the lateral wedges house the vertical expansion wedges during for lateral and angular translation.

FIG. 6B shows the movement of the components when the driver 114 rotates the drive screw 112. As the driver 114 rotates the drive screw 112, the housing 110 translates toward the distal end 118. During this translation, the lateral wedges 106A, 106B expand laterally on the lateral wedge ramps 126, 128, 130 and 132.

FIG. 6C is a sectional top view showing the expandable interbody spacer 100 in an expanded state. The rotation of the drive screw 112 contacts the threaded portion 124 of the housing 110 and translates the housing 110 towards the distal end 118, which forces the lateral wedge 106A, 106B, upper and lower vertical expansion wedges 114A, 114B, 116A, 116B, and upper and lower end plates 102A, 102B, 104A, 104B, outward. The lateral expansion of the expandable interbody spacer 100 stops when lateral expansion limiting balls 136 reaches the limiting groove slots 136.

### Lordosis/Angular Expansion

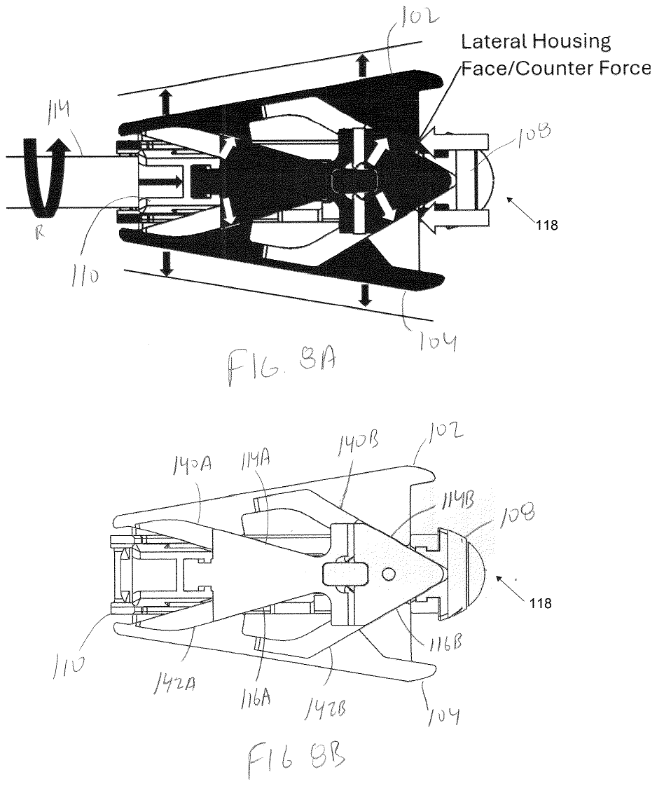

FIGS. 7A-7C are sectional side views showing lordotic expansion of the expandable interbody spacer 100. The upper and lower end plates include distal end plate ramps and proximal end plate ramps. There is a difference in angle between the two ramps which creates the angular/lordotic expansion. When the lateral expansion reaches its limit, the housing begins translating the vertical expansion wedges towards the distal end. At this point the ramps of the vertical expansion wedges contact the angled ramps of the endplates which force them into contact with the proximal housing and distal end creating a counterforce and then they ultimately translate upward.

FIG. 7A is a sectional side view showing the expandable interbody spacer 100 in a collapsed state. The upper end plates 102A, 102B include proximal and distal upper plate ramps 140A, 140B, and the lower end plates 104A, 104B include proximal and distal lower plate ramps 142A, 142B. The proximal and distal upper plate ramps 140A, 140B are configured to engage the proximal and distal upper vertical expansion wedges 114A, 114B, and the proximal and distal lower plate ramps 142A, 142B are configured to engage the proximal and distal lower vertical expansion wedges 116A, 116B. The proximal upper and lower plate ramps 140A, 142A have different angles than the distal upper and lower plate ramps 140B, 142B. In the embodiment shown, the proximal upper and lower plate ramps 140A, 142A include a straight proximal portion (no angle) transitioning to an angled distal portion.

FIG. 7B shows the movement of the components when the driver 114 rotates R the drive screw 112 after lateral expansion. As the driver 114 continues to rotate the drive screw 112, the distal end of the upper and lower endplates 102, 104 contacts a lateral face of the housing 108 creating a counter force F.

The distal upper and lower vertical expansion wedges 114B, 116B translate distally toward the frame 108 and engage the distal upper and lower plate ramps 140B, 142B, lifting the distal ends of the upper and lower endplates 102B, 104B away from each other creating a lordotic angle.

During lordotic expansion, the proximal upper and lower vertical wedges 114A, 116B translate distally without contacting the straight proximal portion of the proximal upper and lower plate ramps 140A, 142A.

FIG. 7C is a sectional side view showing the expandable interbody spacer 100 at maximum lordotic angle. As the drive screw 112 continues to turn, the angle of the proximal plate ramps 140A, 142A and distal plate ramps 140B, 142B will reach a point where their ramp angle matches the angle of the proximal and distal vertical ramps 114, 116. When this point is reached, the lordosis/angular expansion of the endplates stops and the endplates transition to a linear/vertical expansion.

### Linear/Vertical Expansion

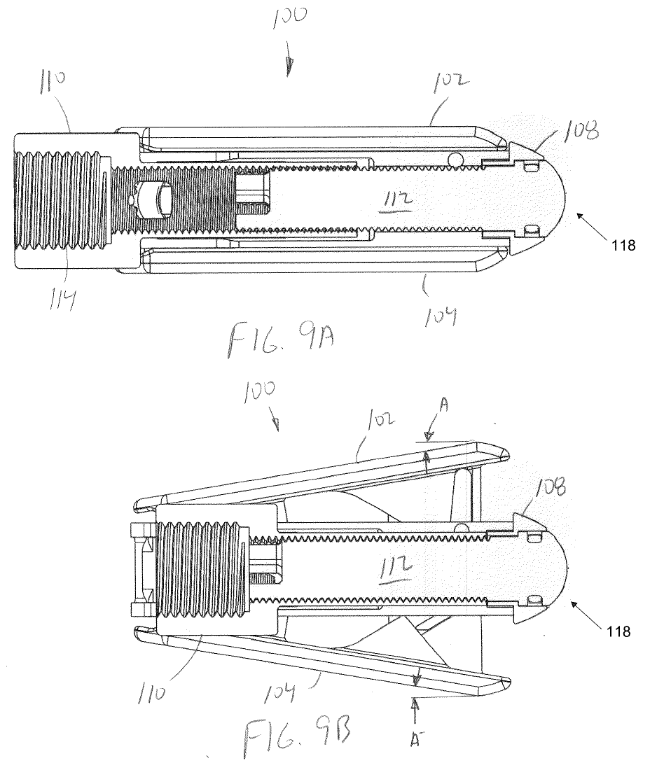

FIGS. 8A, 8B are sectional side views showing linear/vertical expansion of the expandable interbody spacer 100. FIG. 8A shows the movement of the components when the driver 114 rotates R the drive screw 112 after lordosis/angular expansion. At this point, the lordosis/angular expansion is complete and the angle of the proximal plate ramps 140A, 142A and distal plate ramps 140B, 142B match the angle of the proximal and distal vertical ramps 114, 116. As the driver 114 continues to rotate the drive screw 112, the proximal and distal vertical ramps 114, 116 contact the proximal and distal plate ramps 140, 142 to linearly expand the upper and lower endplates 102B, 104B away from each other. Linear/vertical expansion is complete when the distal vertical ramp contacts the frame 108.

FIG. 8B is a sectional side view showing the expandable interbody spacer 100 at maximum linear/vertical expansion of the upper and lower endplates 102, 104.

FIGS. 9A, 9B are sectional side views of the expandable interbody spacer 100 in the collapsed and expanded states.

The upper and lower endplates 102A, 102B, 104A, 104B may include surface features or treatment configured to promote bone growth that engage the bone. For example, the surface may be a textured surface or roughened surface to promote bone integration, or the surface may use a coating or be chemically etched to form a porous or roughened surface. In some embodiments the surface may include teeth. Each of the upper and lower endplates 102A, 102B, 104A, 104B may use the same surface feature or different surface feature.

The expandable interbody spacer 100 components may be fabricated from any biocompatible material suitable for implantation in the human spine, such as metal including, but not limited to, titanium and its alloys, stainless steel, surgical grade plastics, plastic composites, ceramics, bone, or other suitable materials. In some embodiments, surfaces on the components may be formed of a porous material that participates in the growth of bone with the adjacent vertebral bodies. In some embodiments, the components may include a roughened surface that is coated with a porous material, such as a titanium coating, or the material is chemically etched to form pores that participate in the growth of bone with the adjacent vertebra. In some embodiments, only portions of the components may be formed of a porous material, coated with a porous material, or chemically etched to form a porous surface, such as the upper and lower surfaces that contact the adjacent vertebra are roughened or porous.

The expandable interbody spacer 100 may also be used with various tools, such as inserter tools, deployment tools and/or removal tools. The tools may include various attachment features to enable percutaneous insertion of the expandable interbody spacer 100 into the patient. For example, the tools may include arms or clamps to attach to the cutouts or other openings, slots or trenches of the drive mechanism. The tools may also include an actuation device to couple with the rear section of the screw 112. Once the expandable interbody spacer 100 has been inserted and positioned within the intervertebral space between two vertebrae with the insertion tool, the deployment tool may actuate to deploy and expand the expandable interbody spacer 100 by applying a rotational force to screw 112.

In operation, the expandable interbody spacer 100 may be inserted into the intervertebral disc space between two vertebrae using an insertion tool. In some cases, the disc space may include a degenerated disc or other disorder that may require a partial or complete discectomy prior to insertion of the expandable interbody spacer 100. The deployment tool may engage with the rear end of the expandable interbody spacer 100. As the deployment tool applies the rotational force, the expandable interbody spacer 100 gradually expands as described above. The insertion tool is then removed or uncoupled from the expandable interbody spacer 100.

Example embodiments of the methods and systems of the present invention have been described herein. As noted elsewhere, these example embodiments have been described for illustrative purposes only and are not limiting. Other embodiments are possible and are covered by the invention. Such embodiments will be apparent to a person skilled in the relevant art(s) based on the teachings contained herein. Thus, the breadth and scope of the present invention should not be limited by any of the above-described exemplary embodiments but should be defined only in accordance with the following claims and their equivalents.

The invention claimed is:

1. An expandable interbody spacer expanded in sequential steps for placement between adjacent vertebrae comprising:
a proximal housing;
a distal frame;
a drive screw configured to axially translate the proximal housing toward the distal frame;
right and left lateral expansion wedges coupled to the proximal housing and distal frame;
right and left upper endplates;
right and left lower endplates; and
proximal and distal vertical wedges coupled to proximal and distal ends of the right and left upper and lower endplates;
wherein the axial translation of the proximal housing toward the distal frame induces the following sequential steps expanding the expandable interbody spacer:
a) lateral expansion of the right and left expansion wedges by the proximal housing and distal frame, once lateral expansion is complete;
b) lordotic/angular expansion of the distal end of the upper endplates away from the distal end of the lower endplates by the distal vertical wedge resulting in a lordotic angle, once lordotic expansion is complete;
c) linear/vertical expansion of the upper endplates away from the lower endplates by the proximal and distal vertical wedges.

2. The expandable interbody spacer of claim 1, wherein the proximal housing, distal frame and left and right lateral expansion wedges all contain angled ramps configured to induce lateral expansion during axial translation of the proximal housing and distal frame toward each other.

3. The expandable interbody spacer of claim 1, wherein lateral expansion stops when lateral expansion limiting balls reach the end of limiting groove slots.

4. The expandable interbody spacer of claim 1, wherein the right and left upper and lower endplates include proximal and distal upper and lower endplate ramps configured to engage the proximal and distal vertical wedges.

5. The expandable spacer of claim 4, wherein the distal vertical wedge has a steeper angle than the proximal vertical wedge endplate ramps configured to induce lordotic expansion of the distal end of the upper and lower endplate during axial translation.

6. The expandable spacer of claim 5, wherein lordotic expansion stops when the when the proximal and distal plate ramps reach a point where their ramp angle matches the angle of the proximal and distal vertical ramps.

7. The expandable spacer of claim 6, wherein when lordotic expansion stops expansion transitions to linear expansion once the proximal vertical wedge engages the proximal upper and lower endplate ramps.

8. An expandable interbody spacer for placement between adjacent vertebrae comprising:
a proximal housing;
a distal frame;
a drive screw configured to axially translate the proximal housing toward the distal frame;
right and left lateral expansion wedges slidingly coupled to the proximal housing and distal frame;
wherein the proximal housing, distal frame, and left and right lateral expansion wedges all contain angled ramps configured to induce lateral expansion during translation of the proximal housing toward the distal frame;
right and left upper and lower endplates;
proximal and distal vertical wedges coupled to the right and left upper and lower endplates; the distal vertical wedge having a steeper angle than the proximal vertical wedge,
wherein:
initial axial translation induces lateral expansion of the lateral expansion wedges until a lateral expansion stop is reached;
continued axial translation induces lordotic expansion of the right and left upper and lower endplates by the distal vertical wedge until the proximal vertical wedge engages the right and left upper and lower endplate; and
further axial translation induces linear expansion of the right and left upper and lower endplates by both the proximal and distal vertical wedges.

9. The expandable interbody spacer of claim 8, wherein lateral expansion stops when lateral expansion limiting balls reach the end of limiting groove slots.

10. The expandable interbody spacer of claim 8, wherein the vertical expansion wedges are positioned in the slots or cavities of the lateral expansion wedges and laterally translated with the lateral expansion wedges 11. The expandable interbody spacer of claim 8, wherein the right and left upper and lower endplates include proximal and distal upper and lower endplate ramps configured to engage the proximal and distal vertical wedges.

12. The expandable spacer of claim 11, wherein the distal vertical wedge has a steeper angle than the proximal vertical wedge endplate ramps configured to induce lordotic expansion of the distal end of the upper and lower endplate during axial translation.

13. The expandable spacer of claim 12, wherein lordotic expansion stops when the when the proximal and distal plate ramps reach a point where their ramp angle matches the angle of the proximal and distal vertical ramps.

14. An expandable interbody spacer expanded in sequential steps for placement between adjacent vertebrae comprising:
a housing having lateral expansion ramps; and
a frame;
lateral expansion wedges having lateral wedge ramps slidingly coupled to the housing lateral expansion ramps;
a drive screw rotatably coupled to the proximal housing and distal frame, wherein, rotation of the drive screw translates the proximal housing toward the distal frame and induces lateral expansion the right and left lateral expansion wedges away from each other;
right and left upper endplates having proximal and distal endplates ramps;
right and left lower endplates having proximal and distal endplates ramps; and
vertical expansion wedges having proximal vertical ramps configured to slidingly engage the proximal endplate ramps, and distal vertical ramps configured to slidingly engage the distal endplate ramps, wherein:
a difference in angle between the proximal and distal vertical ramps results in angular/lordotic expansion of the upper and lower endplates, and
a matching angle between the proximal and distal vertical ramps results in linear/vertical expansion of the upper and lower endplates;
wherein the axial translation of the proximal housing toward the distal frame induces:
a) lateral expansion of the right and left expansion wedges away from each;
b) once lateral expansion is complete, lordotic/angular expansion of the upper and lower endplates away from each other; and
c) once lordotic expansion is complete. linear/vertical expansion of the upper endplates away from the lower endplates.

15. The expandable interbody spacer of claim 14, wherein the proximal housing, distal frame and left and right lateral expansion wedges contain angled ramps configured to induce lateral expansion during axial translation of the proximal housing and distal frame toward each other.

16. The expandable interbody spacer of claim 14, wherein the vertical expansion wedges are positioned in the slots or cavities of the lateral expansion wedges and laterally translated with the lateral expansion wedges 17. The expandable interbody spacer of claim 14, wherein lateral expansion stops when lateral expansion limiting balls reach the end of limiting groove slots.

18. The expandable spacer of claim 14, wherein the distal vertical wedge has a steeper angle than the proximal vertical wedge endplate ramps configured to induce lordotic expansion of the distal end of the upper and lower endplate during axial translation.

19. The expandable spacer of claim 18, wherein lordotic expansion stops when the when the proximal and distal plate ramps reach a point where their ramp angle matches the angle of the proximal and distal vertical ramps.

20. The expandable spacer of claim 19, wherein when lordotic expansion stops expansion transitions to linear expansion once the proximal vertical wedge engages the proximal upper and lower endplate ramps.
